(19) United States
(12) Patent Application Publication
(10) Pub. No.: US 2026/0335614 A1
(43) Pub. Date: Oct. 29, 2026

(54) CEREBROSPINAL FLUID DRAINAGE DEVICE AND METHOD

(71) Applicant: Loh Medical Holdings LLC, Seattle, WA (US)

(72) Inventor: Yince Loh, Seattle, WA (US)

(73) Assignee: Loh Medical Holdings LLC, Seattle, WA (US)

(21) Appl. No.: 19/654,922

(22) Filed: Apr. 22, 2026

Related U.S. Application Data
(60) Provisional application No. 63/793,506, filed on Apr. 23, 2025.

(51) Int. Cl. A61M 27/00

(52) U.S. Cl. CPC A61M 27/006; A61M 2027/004

(57) ABSTRACT

A percutaneously implantable cerebrospinal fluid draining device that drains cerebrospinal fluid from the subarachnoid space to the venous plexus to provide hydrocephalus relief. The device can be installed without an intricate, complex open surgery.

### PRIORITY DATA

This application claims priority from the following U.S. patent application: U.S. Provisional Application Ser. No. 63/793,506, filed on Apr. 23, 2025. The disclosure of such application is incorporated herein, by reference, in its entirety.

### BACKGROUND

Cerebrospinal fluid (“CSF”) is essential to buoy the brain inside the skull. CSF is made by the choroid plexus and, through the arachnoid granulations, CSF is absorbed into the venous blood system. Pathological conditions may sometimes increase the production of CSF or decrease its absorption, resulting in a condition called hydrocephalus. The mainstay of hydrocephalus treatment is CSF diversion with shunts. Existing shunt systems to treat hydrocephalus require intricate, complex open surgery and are prone to failing. These existing shunt systems can also become a focus of infection, which may often require further surgery.

Accordingly, there is a need for a CSF draining device and method that is easily insertable into a patient to provide hydrocephalus relief without an intricate, complex open surgery.

### SUMMARY

In one aspect, a drainage device is provided that includes a distal anchoring structure, a proximal anchoring structure, and a connecting section extending between the anchoring structures and defining a lumen. Each anchoring structure comprises a plurality of discrete prongs. The prongs are configured to be constrained during delivery and to expand upon deployment, such as by elastic recovery of a shape memory material. In a deployed configuration, the distal anchoring structure engages a distal side of a dorsal dural border and the proximal anchoring structure engages a proximal side of the dorsal dural border, thereby securing the device across an opening formed in the dorsal dural border. The lumen of the connecting section provides fluid communication between the subarachnoid space and the venous system to permit drainage of cerebrospinal fluid.

In certain embodiments, the prongs are independently movable, circumferentially spaced, and define gaps therebetween. The prongs may extend in three-dimensional, non-planar configurations and may be arranged symmetrically or asymmetrically. The prongs may be biased to extend in opposing axial directions on the distal and proximal anchoring structures and may be configured to exert pressure on adjacent tissue to secure the device. The device may be formed of a shape memory alloy, such as medical grade nitinol, allowing transition between constrained and expanded states.

In another aspect, a method of treating hydrocephalus is provided. The method includes percutaneously advancing a needle along a posterior spinal access path through a venous region, through a dorsal dural border, and into a subarachnoid space. A drainage device is introduced through the needle in a constrained configuration. A distal anchoring structure is advanced beyond a distal opening of the needle and deployed within the subarachnoid space. The needle is then withdrawn relative to the device to expose and deploy a proximal anchoring structure within the venous region. In the deployed configuration, the distal and proximal prongs engage opposing sides of the dorsal dural border to secure the device, and cerebrospinal fluid is drained through the lumen into the venous region. In certain embodiments, deployment occurs solely by relative movement between the needle and the device, without use of a sheath retraction mechanism.

In a further aspect, a system for implanting the drainage device is provided. The system includes a delivery needle having a hollow outer shaft defining a lumen, the drainage device positioned within the lumen, and a pusher element configured to advance the device through the needle. The needle constrains the prongs during insertion. The distal anchoring structure is configured to deploy upon exiting a distal opening of the needle, and the proximal anchoring structure is configured to deploy upon withdrawal of the needle. The prongs may be constrained directly by contact with an inner wall of the needle without use of a separate sheath.

### DRAWINGS

Objects, features, and advantages of the present invention will become apparent upon reading the following description in conjunction with the drawing figures, in which:

FIG. 1 is a perspective view of an embodiment of a cerebrospinal fluid drainage device of the present invention;

FIG. 2 is a side view of the embodiment of the present invention depicted in FIG. 1;

FIG. 3 is a left, end view of the embodiment of the present invention depicted in FIG. 2;

FIG. 4 is a side view of an embodiment of a cerebrospinal fluid drainage device of the present invention, where a cerebrospinal fluid drainage device is disposed and compressed within a retaining sheath;

FIG. 5A is a side view of an embodiment of an outer shaft of a needle for use with a cerebrospinal fluid drainage device of the present invention;

FIG. 5B is a side view of an embodiment of a stylet of a needle for use with a cerebrospinal fluid drainage device of the present invention;

FIG. 6A is a side view of an embodiment of an assembled needle for use with a cerebrospinal fluid drainage device of the present invention;

FIG. 6B is a front view of an embodiment of an assembled needle for use with a cerebrospinal fluid drainage device of the present invention;

FIG. 7 depicts an area of interest of a spinal cord of a patient where a cerebrospinal fluid drainage device of the present invention may be implanted;

FIG. 8A is a partial cross-sectional view schematically illustrating a step in the implantation of a cerebrospinal fluid drainage device of the present invention in a subarachnoid space of a patient;

FIG. 8B is a view similar to FIG. 8A, but illustrating a subsequent step of the implantation of a cerebrospinal fluid drainage device of the present invention in a subarachnoid space of a patient;

FIG. 8C is a view similar to FIG. 8B, but illustrating a subsequent step of the implantation of a cerebrospinal fluid drainage device of the present invention in a subarachnoid space of a patient;

FIG. 8D is a view similar to FIG. 8C, but illustrating a subsequent step of the implantation of a cerebrospinal fluid drainage device of the present invention in a subarachnoid space of a patient;

FIG. 8E is a view similar to FIG. 8D, but illustrating a subsequent step of the implantation of a cerebrospinal fluid drainage device of the present invention in a subarachnoid space of a patient;

FIG. 8F is a view similar to FIG. 8E, but illustrating a subsequent step of the implantation of a cerebrospinal fluid drainage device of the present invention in a subarachnoid space of a patient;

FIG. 8G is a magnified view of the area of interest in FIG. 8F, depicting an embodiment of a cerebrospinal fluid drainage device of the present invention implanted in the body across the dura of a patient; and

FIG. 9 is a side view of an alternative embodiment of a stylet of a needle for use with a cerebrospinal fluid drainage device of the present invention.

### DESCRIPTION

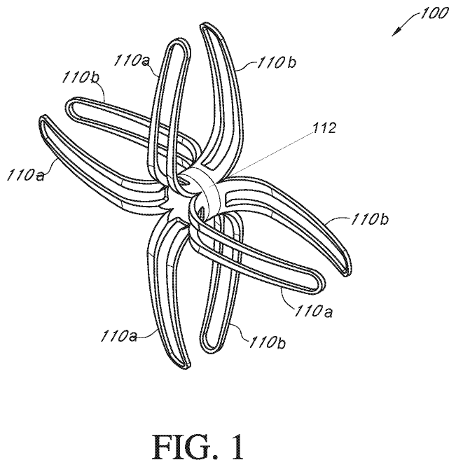

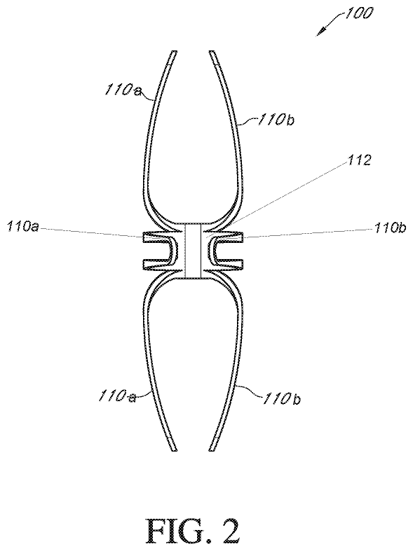

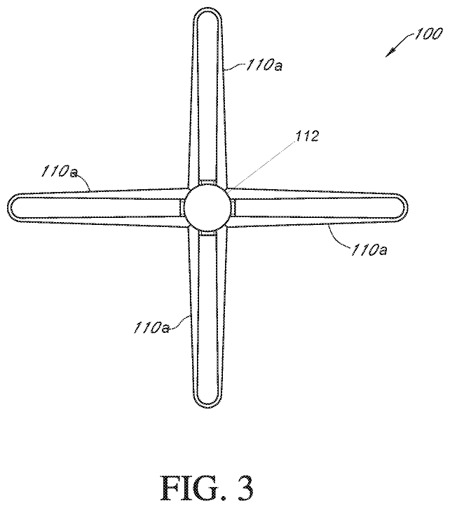

Referring to FIGS. 1-3, an embodiment of a CSF drainage device 100 of the present invention is depicted. The CSF drainage device 100 of the embodiment depicted includes a first set of prongs 110a, a second set of prongs 110b and a center connecting section 112 that are all integrated with one another so as to create a portal or lumen between the first set of prongs 110a and the second set of prongs 110b. In this embodiment, the first set of prongs 110a are four prongs formed on one side of the center connecting section 112, and the second set of prongs 110b are four prongs formed on the opposite side of the center connecting section 112. The prongs 110a, 110b may be made from any material that is medical grade that is compressible and that expands, when decompressed, to re-form and maintain a rigid structure (e.g., a shape memory alloy material). In this embodiment, the prongs 110a, 110b are made of medical grade nitinol: a nickel and titanium alloy.

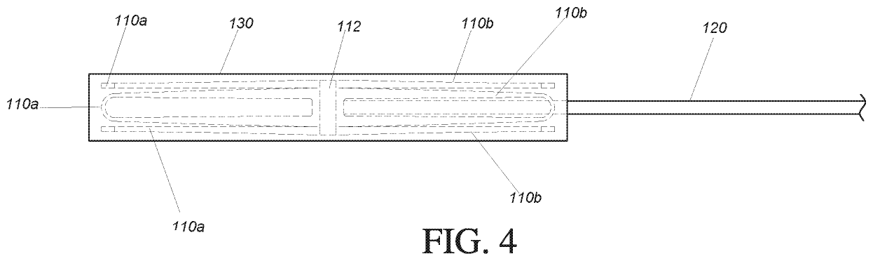

Referring now to FIG. 4, in conjunction with FIGS. 1-3, because the prongs 110a, 110b are made from a resilient material, the CSF drainage device 100 is moveable between a fully expanded state as illustrated in FIGS. 1-3, and a compressed state as illustrated in FIG. 4. In this embodiment, when stored, and for transport, the CSF drainage device 100 is compressed and inserted into a retaining sheath 130. The retaining sheath 130 keeps the CSF drainage device 100 in a compressed state until ready for use (FIG. 4). Further, the CSF drainage device 100 is connected to a pusher wire 120. The CSF drainage device 100 may be connected to the pusher wire 120 in many different known ways, such as using a mechanical or electrolytic mechanism.

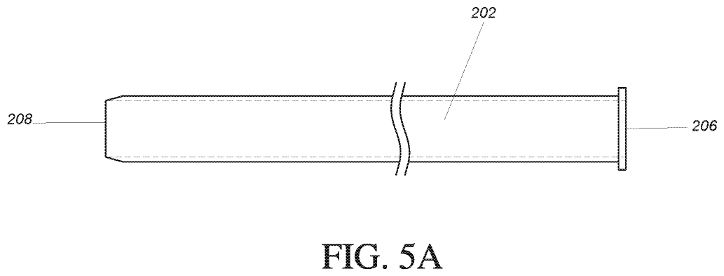

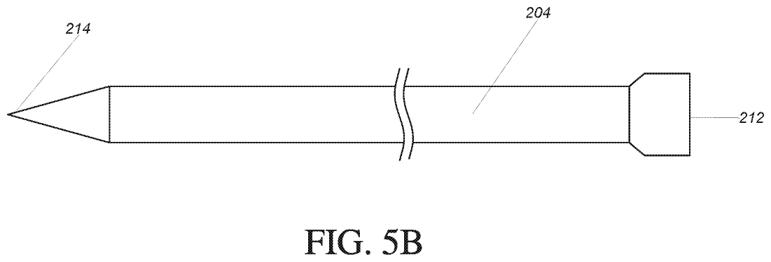

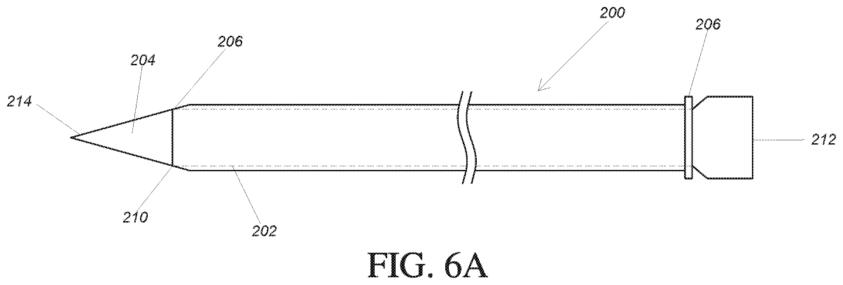

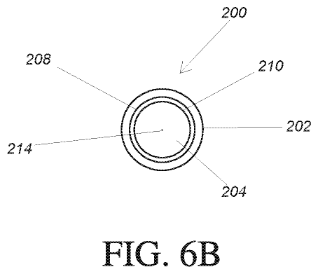

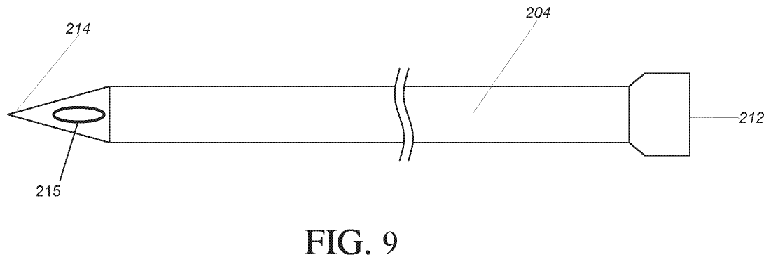

Referring to FIGS. 5A, 5B, 6A and 6B, an embodiment of a needle 200 (FIG. 6A) and its components are depicted. The needle 200 can be used, as described in more detail below, to implant the CSF drainage device 100 percutaneously. Any known assembled needle configuration or implanting system can be used to implant the CSF drainage device 100. The needle 200 of one depicted embodiment has two components: a hollow outer shaft 202 (FIG. 5A) and a solid stylet 204 (FIG. 5B). In another embodiment depicted in FIG. 9, the stylet 204 has a hollow interior. The hollow outer shaft 202 has an open back end hub 206 and a open front end 208 that have an inner lumen between them. When the outer shaft 202 is inserted into a body of a patient, the back end hub 206 is proximal, and the front end 208 is distal. The front end 208, in this embodiment, has a concentric taper and forms a circular opening 210 (FIG. 6B). In preferred embodiments, the outer shaft 202 is formed having an outer width narrow enough to advance into the back of a patient and be advanced across the dura mater of the spinal canal while imparting minimal trauma, while also being formed to have an inner lumen wide enough to allow the constrained CSF drainage device 100 to be advanced through the outer shaft 202.

The stylet 204, in the one embodiment depicted in FIG. 5B, has a back end 212 and a concentric, pointy front end 214. When the stylet 204 is inserted all the way into the outer shaft 202 in the described embodiment, the stylet 204 lies coaxially within the outer shaft 202, and the two combined components form the assembled needle 200 (FIGS. 6A-6B), and the concentric, pointy front end 214 is shaped so that the front end 214 extends the concentric taper of the front end 208 of the outer shaft 202. Due to the outer shaft 202 of the needle 200 having an even, circular opening at the front end 208, the embodiment of the needle 200 described herein is effective for use with the CSF drainage device 100 because it allows the first set of prongs 110a to expand simultaneously when deployed. In another embodiment of the stylet 204, depicted in FIG. 9, the stylet 204 is hollow throughout the interior of the stylet 204 creating a lumen therein. A side port 215 is formed in the pointy, front end 214 and is in communication with a hollow back end 212 of the stylet 204. A syringe can be attached to the hollow back end 212 to deliver fluid or medicine to the front end 214 of the stylet 204 through the side port 215.

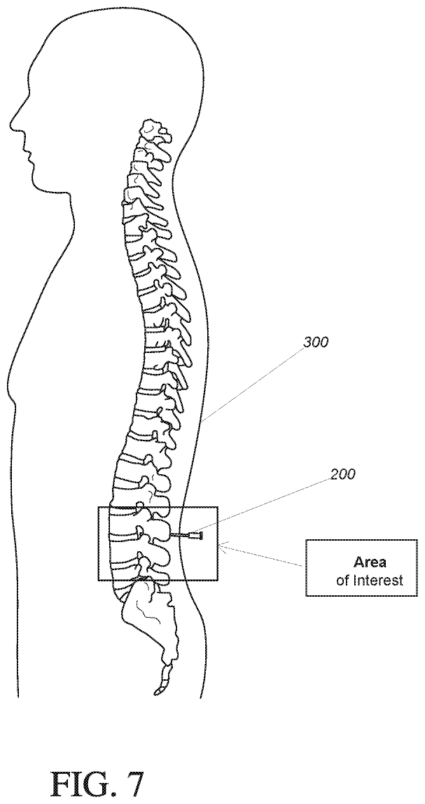

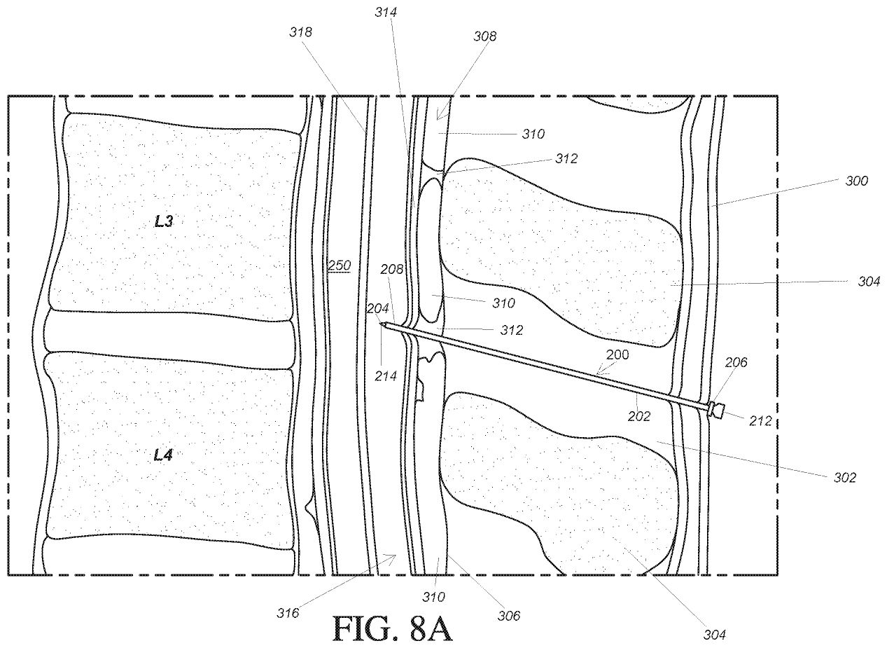

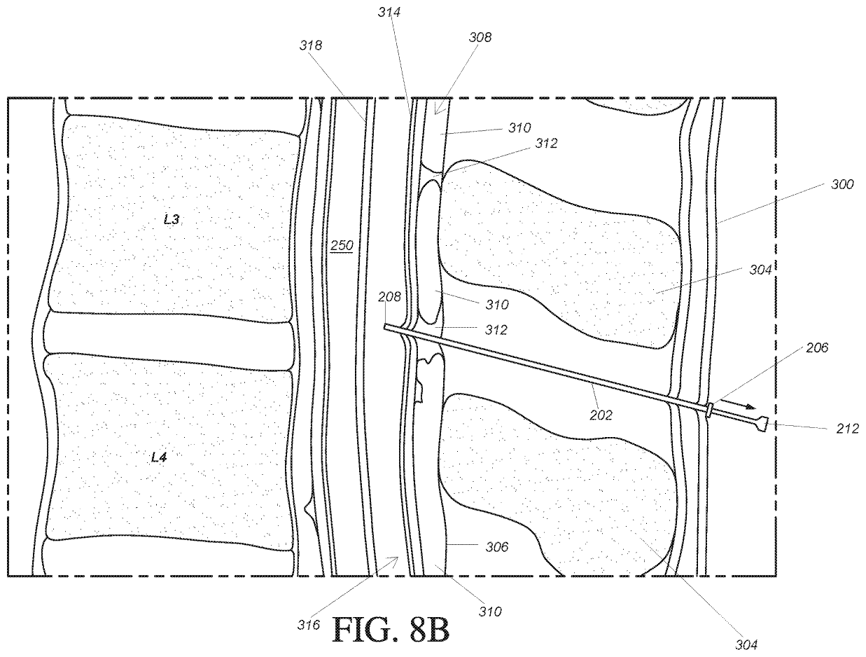

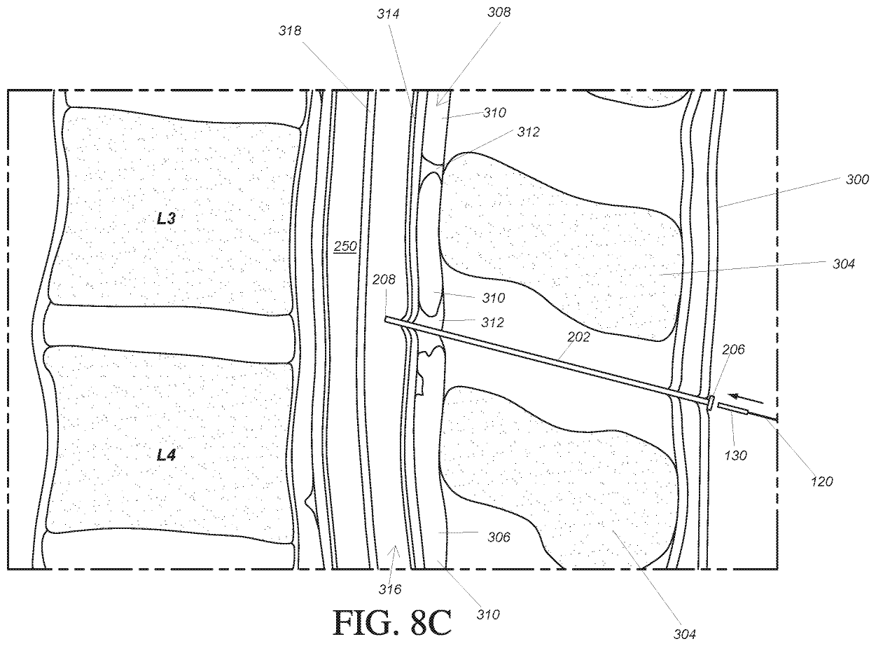

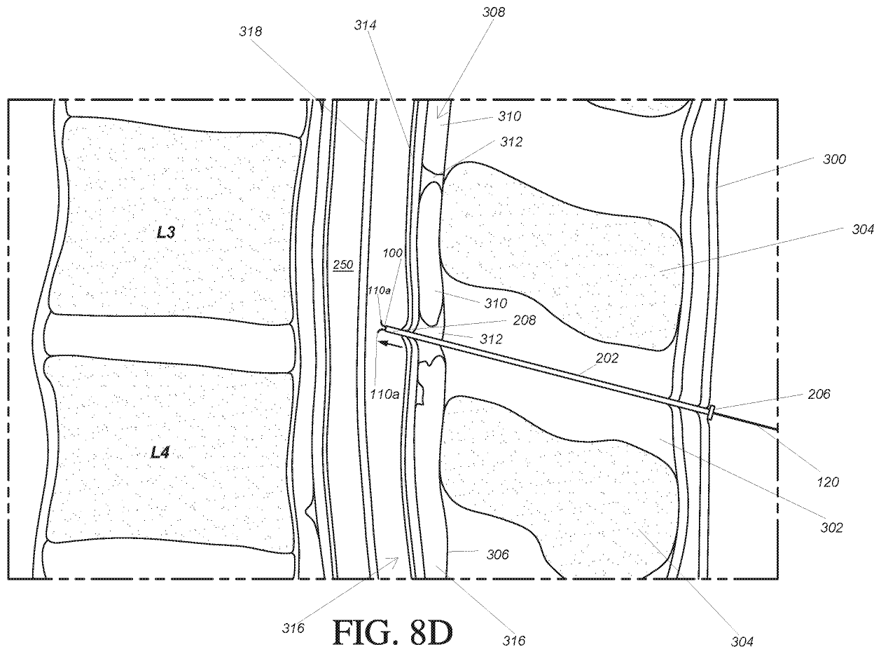

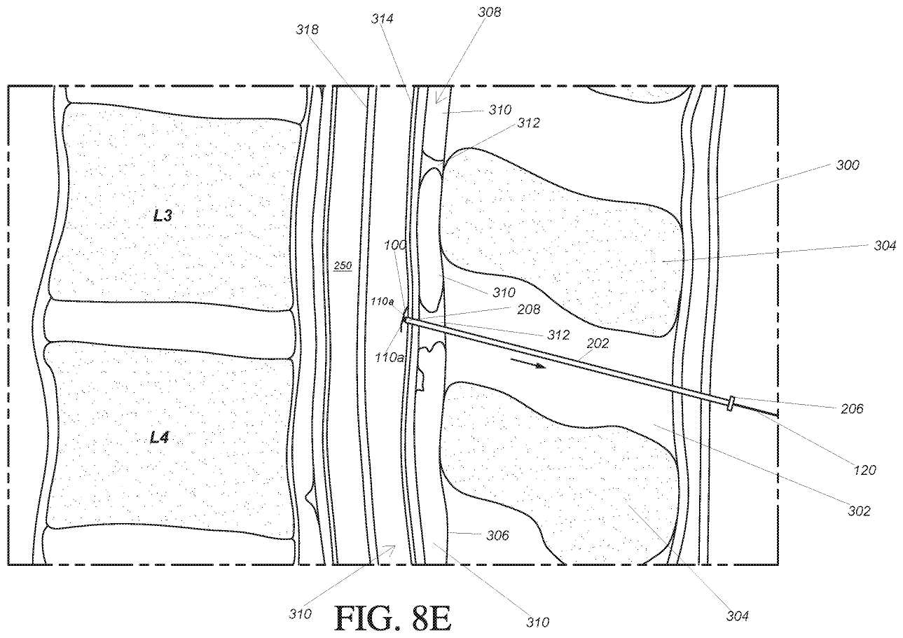

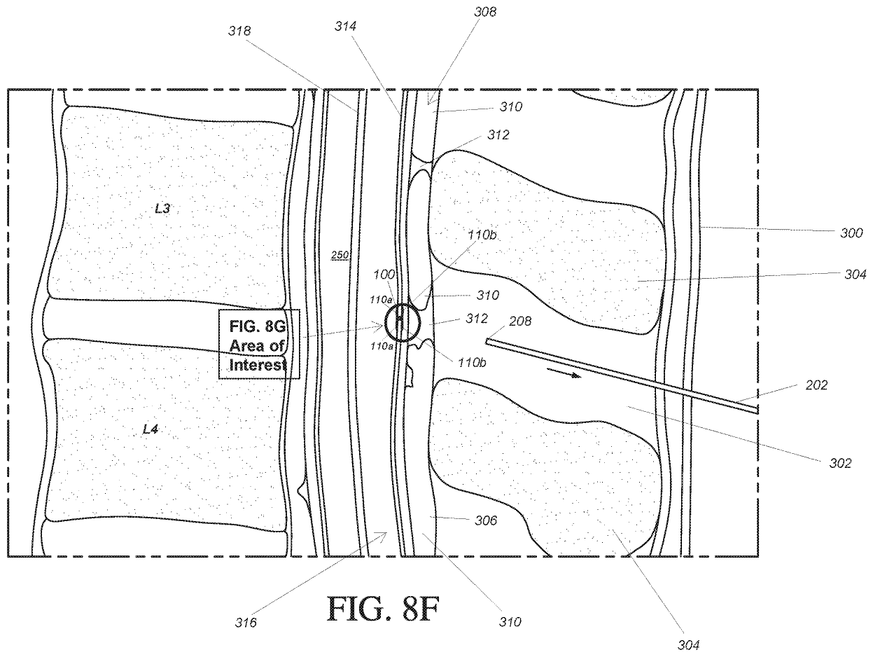

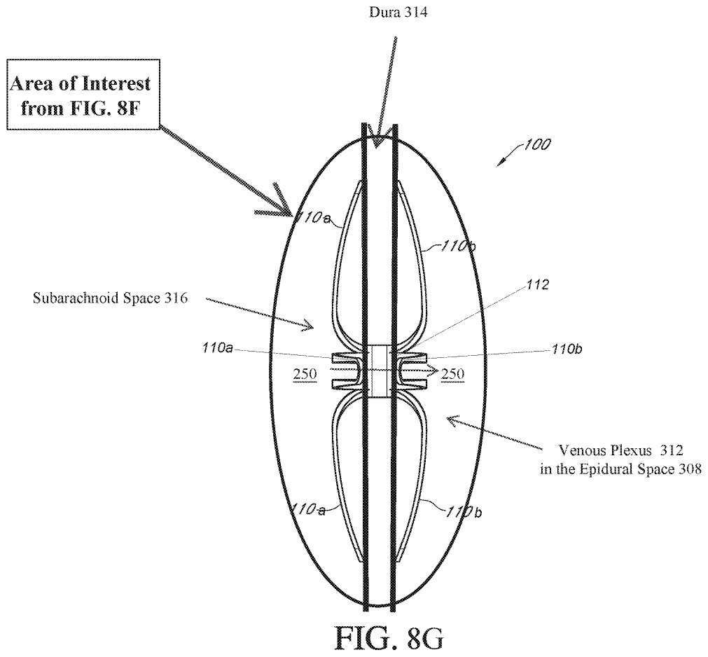

Referring now to FIGS. 7 and 8A-8G, an exemplary method of percutaneously implanting the CSF drainage device 100, using the assembled needle 200 to use the CSF drainage device 100 to catheterize the inside of the cerebrospinal fluid column beyond the dura of the spinal canal of the patient, is depicted. The exemplary method does not require open surgery. Referring specifically to FIG. 8A, the first step is to insert the assembled needle 200 inserted into the skin 300 of the back of the patient and advanced the needle 200 into the interspinous ligament 302 between the spinous processes 304. The pointy front end 214 of the stylet 204 pierces the interspinous ligament 302, and other portions of the body, which allows the surgeon or medical practitioner to advance the needle 200 through the patient's back as described below. The surgeon or medical practitioner continues to advance the needle 200 through the interspinous ligament 302, through the ligamentum flavum 306 into the epidural space 308. While advancing, in one embodiment, the practitioner gently pushes on a syringe attached to the back end 212 of the stylet 204, which contains air or the patient's own blood. In FIGS. 7 and 8A-8G, the needle 200 is depicted in the interspace between the third and fourth lumbar vertebrae. It should be understood, though, this method can be applied to any spinal level.

The epidural space 308 includes epidural fat 310 and the internal venous plexus 312. When advancing the needle 200 and, in one embodiment, the attached syringe through the epidural space 308, the surgeon or medical practitioner wants to make sure the needle 200 passes through the internal venous plexus 312 because, when put in place, the CSF drainage device 100 will drain into the internal venous plexus 312. As is known in the art, a surgeon or medical practitioner will feel for pressure in this area and feel a loss of resistance to the gentle pressure placed on the syringe to ensure that the needle 200 has passed through the internal venous plexus 312 and not just epidural fat 310. With the needle 200 passing through the internal venous plexus 312, the surgeon or medical practitioner continues to advance the needle 200 through the dorsal dural border 314 into the subarachnoid space 316, short of the cauda equina 318. With the needle 200 in the subarachnoid space 316, the surgeon or medical practitioner has now accessed the space where the CSF 250 resides.

Referring now to FIG. 8B, the surgeon or medical practitioner now starts to withdraw the stylet 204 from within the outer shaft 202. The outer shaft 202 stays in place to create a pathway to insert the CSF drainage device 100, and the surgeon or medical practitioner observes the outflow of CSF 250. As depicted in FIG. 8C, the surgeon or medical practitioner, then, inserts the CSF drainage device 100 into the proximal end 206 of the outer shaft 202 and removes the sheath 130. Once the sheath 130 is removed, the CSF drainage device 100 naturally expands a little until the CSF drainage device 100 touches the inner sidewalls of the outer shaft 202. The CSF drainage device 100 is still compressed at this point. With the CSF drainage device 100 inside the outer shaft 202, the surgeon or medical practitioner then advances the CSF drainage device 100 through the outer shaft 202, using the pusher wire 120, until the CSF drainage device 100 is at the front end 208 of the outer shaft 202, past the dorsal dural border 314.

Now, as depicted in FIG. 8D, the surgeon or medical practitioner continues to advance the CSF drainage device 100 with the pusher wire 120. As the CSF drainage device 100 starts to emerge from the outer shaft 202, the first set of prongs 110a (the distal prongs) start to naturally expand inside the subarachnoid space 316, and in this embodiment, the first set of prongs 110a expand symmetrically since the front end 208 of the outer shaft 202 defines a circular opening 210. In one embodiment, the surgeon or medical practitioner can feel a click when the distal prongs 110a fully expand. As depicted in FIG. 8E, once the first set of prongs 110a are fully expanded, the surgeon or medical practitioner continues to pull the outer shaft 202 proximally towards him. As the outer shaft 202 is pulled proximally, together with the pusher wire 120, the expanded distal prongs 110a engage the subarachnoid side of the dorsal dural border 314. The engagement of the expanded distal prongs 110a with the dorsal dural border 314 acts as a stay within the inside of the dorsal dural border 314, allowing force to be applied to the outer shaft 202 to withdraw the outer shaft 202 coaxially over the pusher wire 120 from the patient's body without pulling the CSF drainage device 100 through the hole made in the dorsal burial border 314. As the outer shaft 202 continues to be pulled proximally, the second set of prongs 110b (the proximal prongs) naturally expands symmetrically as the proximal prongs 110b are unsheathed from the front end 208 of the outer shaft 202. In one embodiment, as with the distal prongs 110a, the surgeon or medical practitioner can feel a click when the proximal prongs 110b fully expand. As depicted in FIGS. 8F and 8G, once the proximal prongs 110b are fully expanded on the posterior side of the dorsal dural border 314, the CSF drainage device 100 is fully deployed and locked in place. In the fully deployed configuration, the proximal prongs 110b are directly opposed to the paired distal prongs 110a, and each pair of proximal and distal prongs 110a, 110b spring towards each other on opposite sides of the dorsal dural border 314. In this configuration, each pair of proximal and distal prongs 110a, 110b “trap” or “sandwich” the dorsal dural border 314 between them, anchoring the CSF drainage device 100 in place with a tight fit. The pusher wire 120 can now be removed from the CSF drainage device 100, either through a mechanical, electrolytic or other known detachment mechanism.

With the CSF drainage device 100 in place, physiologic drainage of CSF 250 can occur. The CSF 250 in the subarachnoid space 316 now flows through the lumen formed by the center connecting section 112 of the CSF drainage device 100 directly into the internal venous plexus 312, in the same manner that CSF 250 re-absorption occurs naturally in people with normal levels of CSF 250. The method and device disclosed herein has the further advantage over surgery by allowing for the placement of multiple CSF drainage devices 100 if needed to create more fluid diversion in the case of under-drainage, and in the case of over-drainage, providing the ability to deactivate a CSF drainage device 100 if needed using a fluoroscopically-guided percutaneous liquid embolization or traditional “blood patching” performed routinely for CSF leakage.

Other embodiments might include different methods of expanding and constraining the distal portion of the CSF drainage device 100. Other embodiments might include different methods of expanding and constraining the proximal portion of the CSF drainage device 100.

Other embodiments might also include distal portions of the anchor wire and wire shaft itself to be composed of non-metallic or bioabsorbable material. Other embodiments might also include proximal portions of the anchor wire and wire shaft itself to be composed of non-metallic or bioabsorbable material. In another embodiment the nonmetallic distal portion may be disc-shaped and compressible to fit in the delivery catheter yet expand to a full disc shape once inside the dural border in order to act as an anchor. In such an embodiment, the proximal anchor would similar be disc shaped and similar to the pronged embodiment, achieve an anchored position by “sandwiching” dura between the proximal and distal anchors. Other embodiments may provide for a one-way valve such as a ball-valve mechanism to reside in the connecting section 112.

Although certain embodiments and features of a cerebrospinal fluid drainage device and methods for use have been described herein, the scope of coverage of this patent is not limited thereto. On the contrary, this patent covers all embodiments of the teachings of the disclosure that fairly fall within the scope of permissible equivalents.

What is claimed is:

1. A device for draining cerebrospinal fluid from a subarachnoid space of a patient into the venous system of the patient through an opening formed in the dorsal dural border of the patient, the device comprising:
a first set of prongs;
a second set of prongs; and
a center connecting section which connects the first set of prongs on one side of the center connecting section to the second set of prongs on the opposite side of the center connecting section to form a lumen:
wherein the first set of prongs, the second set of prongs and the center connecting section each comprise a shape memory alloy material, allowing the device to be in a compressed state when being inserted into the body of the patient and to be deployed into an expanded state for use in the body of the patient;
wherein in the expanded state in the body of the patient, the expanded first set of prongs engage with a distal side of the dorsal dural border and the expanded second set of prongs engage with a proximal side of the dorsal dural border to anchor the device between the subarachnoid space and the venous system in the opening formed in the dorsal dural border; and further wherein, the lumen of the center connecting section is disposed within the opening formed in the dorsal dural border and puts the cerebrospinal fluid from the subarachnoid space in fluid communication with the venous system to allow the cerebrospinal fluid from the subarachnoid space to drain into the venous system.

2. The drainage device of claim 1, wherein each prong is independently movable relative to adjacent prongs.

3. The drainage device of claim 1, wherein the prongs are circumferentially spaced and define gaps therebetween in both constrained and deployed configurations.

4. The drainage device of claim 1, wherein each prong has a distal tip configured to exert pressure on the dorsal dural border.

5. The drainage device of claim 1, wherein the prongs deploy by flexing from a longitudinally aligned configuration.

6. The drainage device of claim 1, wherein the prongs are biased to extend in opposing axial directions on the distal and proximal anchoring structures.

7. The drainage device of claim 1, wherein the prongs provide fixation of the drainage device through pressure on both sides of the dorsal dural border.

8. The drainage device of claim 1, wherein the first and second set of prongs each have four discrete prongs.

9. The drainage device of claim 1, wherein the prongs are non-planar and extend in three-dimensional space.

10. The drainage device of claim 1, wherein the prongs are asymmetrically arranged around the connecting member.

11. The drainage device of claim 1, wherein the drainage device is made of a shape memory alloy material.

12. The drainage device of claim 1, wherein the drainage device is made of a medical grade nitinol alloy.

13. A method of treating hydrocephalus in a patient, comprising:
percutaneously advancing a needle through a posterior spinal access path through a venous region, through a dorsal dural border and into a subarachnoid space, adjacent the dorsal dural border;
introducing a drainage device for cerebrospinal fluid through the needle in a constrained configuration; wherein the drainage device includes connected, opposing distal and proximal anchoring structures with each anchoring structure having a plurality of discrete prongs and the connection between the anchoring structures forming a lumen;
advancing the distal anchoring structure beyond a distal opening of the needle;
deploying the plurality of discrete prongs on the distal anchoring structure within the subarachnoid space;
withdrawing the needle relative to the drainage device to expose and deploy the plurality of discrete prongs on the proximal anchoring structure within the venous region;
in the deployed configuration, allowing the plurality of discrete prongs of the distal anchoring structure to extend and exert pressure on adjacent tissue of the distal dorsal dural border and the plurality of discrete prongs of the proximal anchoring structure to extend and exert pressure on adjacent tissue of the proximal dorsal dural border to secure the drainage device across the dorsal dural border; and
with the draining device deployed, draining cerebrospinal fluid from the subarachnoid space through the lumen of the draining device into the venous region.

14. The method of claim 13, wherein deployment of the plurality of discrete prongs on both the proximal and distal anchoring structures occurs solely by relative movement between the needle and the drainage device without expansion by an outer restraining structure.

15. The method of claim 13, wherein the plurality of discrete prongs on the distal anchoring structure deploy before the plurality of discrete prongs on the proximal anchoring structure without use of a sheath retraction mechanism.

16. A system for implanting a cerebrospinal fluid drainage device through a posterior spinal access path through a venous region, through a dorsal dural border and into a subarachnoid space, adjacent the dorsal dural border, comprising:
a delivery needle having a hollow outer shaft defining a lumen extending between a proximal end and a distal opening;
a drainage device positioned within the lumen of the delivery needle, the drainage device comprising:
a distal anchoring structure having a plurality of discrete prongs,
a proximal anchoring structure having a plurality of discrete prongs, and
a connecting section defining a fluid lumen; and
a pusher element configured to advance the drainage device through the lumen of the delivery needle,
wherein:
the delivery needle constrains the plurality of discrete prongs of both the distal anchoring structure and the proximal anchoring structure in a constrained configuration during insertion;
the distal anchoring structure is configured to deploy upon exiting the distal opening of the needle;
the proximal anchoring structure is configured to deploy upon withdrawal of the needle and exiting the proximal end of the needle; and
the plurality of discrete prongs of the distal and proximal anchoring structures both deploy by elastic deformation into tissue-engaging positions.

17. The system of claim 16, wherein the prongs of the drainage device are constrained by direct contact with an inner wall of the hollow outer shaft without use of a separate sheath.

18. The system of claim 16, wherein the prongs of the drainage device are circumferentially spaced and define gaps therebetween in both constrained and deployed configurations.

19. The system of claim 16, wherein each prong of the drainage device has a distal tip configured to exert pressure on the dorsal dural border.

20. The system of claim 16, wherein the drainage device is made of a medical grade nitinol alloy.
